(19) United States
(12) Patent Application Publication
(10) Pub. No.: US 2003/0168520 A1
(43) Pub. Date: Sep. 11, 2003

(54) Emanator for evaporation of a liquid therefrom

(76) Inventors: Carl Triplett, Scottsdale, AZ (US); Kristopher J. Stathakis, Scottsdale, AZ (US)

Correspondence Address: Deborah K. Henscheid, Esq., Snell & Wilmer, L.L.P., One Arizona Center, 400 East Van Buren, Phoenix, AZ, 85004-2202, US

(21) Appl. No.: 10/091,256

(22) Filed: Mar. 5, 2002

(51) Int. Cl. A61L 9/04

(52) U.S. Cl. 239/44

(57) ABSTRACT

An emanator for evaporation of a liquid therefrom is provided. The emanator includes a first material and a second material that is disposed adjacent to the first material. The emanator is configured such that a liquid travels through the emanator at a rate no less than a rate at which it would travel through the first material alone and no less than a rate at which it would travel through the second material alone.

### TECHNICAL FIELD

The present invention relates generally to an emanator for evaporation of a liquid, and more particularly to an air freshener emanator for wicking and evaporation of a liquid fragrance.

### BACKGROUND

Air fresheners function by emitting or dispensing liquid fragrances into the air. In certain air freshener designs, the liquid fragrances are formed of fragrance materials, such as perfumes, that are suspended in a liquid carrier which evaporates into the air. The evaporation rate of the liquid carrier may depend on a number of environmental conditions not related to the air freshener itself, such as temperature, humidity, and ambient air flow, and physical properties of the liquid fragrances, such as volatility.

While evaporation of the liquid fragrance can take place directly from the surface of a bulk source of the liquid fragrance, the effectiveness of the air freshener can be greatly enhanced when the liquid fragrance is supplied to and dispersed by an emanator. An emanator is a structure in the air freshener that provides a surface area from which the liquid fragrance may evaporate. Typically, the ability of the liquid fragrance to flow through the emanator and to evaporate from the emanator is a function of the materials used to form the emanator and a function of the configuration of those materials. The effectiveness of an air freshener can be adversely affected by an emanator formed of materials that absorb the liquid fragrance, thereby trapping the liquid fragrance in the emanator, an emanator formed of materials and configured so that flow of the liquid fragrance through the emanator is relatively slow, or an emanator formed of materials that retain excessive residual fragrance materials.

Accordingly, an emanator formed of materials and configured to enhance the movement of liquid fragrance through the emanator has long been needed. An emanator formed of materials and configured to enhance the evaporation of the liquid fragrance from the emanator has also long been needed.

### SUMMARY OF THE INVENTION

While the way in which the invention addresses these long felt needs will be described in greater detail below, this summary of the invention section is intended to introduce the reader to aspects of the invention and is not a complete description of the invention. Particular aspects of the invention are pointed out in other sections herein below, and the invention is set forth in the appended claims which alone demarcate its scope.

In accordance with an exemplary embodiment of the present invention, an emanator for evaporation of a liquid therefrom is provided. The emanator includes a first material and a second material disposed adjacent to the first material. The emanator is configured such that a liquid travels through the emanator at a rate no less than a rate at which it would travel through the first material alone and no less than a rate at which it would travel through the second material alone.

In accordance with another exemplary embodiment of the present invention, an air freshener comprises a source of a liquid fragrance and an emanator in fluid communication with the source of liquid fragrance. The emanator includes a first material through which the liquid fragrance has a first travel rate. The emanator also includes a second material through which the liquid fragrance has a second travel rate. The second material contacts the first material. The emanator is configured such that the liquid fragrance has a third travel rate through the emanator. The third travel rate is no less than the first travel rate and the second travel rate.

In accordance with a further exemplary embodiment of the invention, an emanator for evaporation of a liquid therefrom includes a first material and a second material adjacent to the first material and forming an interface with the first material. The emanator is configured to permit a liquid to travel along the interface and to evaporate from at least one of the first material and the second material.

In yet another exemplary embodiment of the invention, a refill for an air freshener is provided. The refill includes a source of a liquid fragrance and an emanator in contact with the source of the liquid fragrance. The emanator comprises a first material and a second material contacting the first material. The emanator is configured such that the liquid fragrance travels through the emanator at a rate no less than a rate at which it would travel through the first material alone and no less than a rate at which it would travel through the second material alone.

### BRIEF DESCRIPTION OF THE DRAWINGS

A more complete understanding of the present invention may be derived by referring to the detailed description and claims, considered in connection with the figures, wherein like reference numbers refer to similar elements throughout the figures, and:

FIG. 1 is a cross-sectional view of an air freshener in accordance with an exemplary embodiment of the present invention; and

FIG. 2 is a cross-sectional view of an air freshener in accordance with another exemplary embodiment of the present invention.

### DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

The present invention is directed to an emanator for the evaporation of volatile fluids, such as, for example, liquid fragrances. In an exemplary embodiment of the invention, the emanator is used in an air freshener. It will be appreciated, however, that the emanator is not limited to use in air fresheners and may be used in any other suitable device for the evaporation of liquids.

In one exemplary embodiment of the invention, the emanator comprises a first material and a second material that is disposed adjacent to the first material. The emanator is configured such that a liquid travels through the emanator at a rate faster than or equal to the rate it would travel through the first material alone or the second material alone. In other words, the first material typically permits a liquid to travel therethrough at a first travel rate. As used herein, the term “travel rate” means the rate at which the liquid is absorbed by a material or otherwise flows or moves through the material. The second material typically permits the liquid to travel therethrough at a second travel rate. The emanator is configured such that the liquid has a third travel rate through the emanator, where the third travel rate is faster than or equal to the first or second travel rates. In this manner, the emanator may benefit from the physical, chemical or electrical properties of each of the individual materials. By way of example, one material may exhibit a particular rigidity that gives the emanator a desired stiffness while the other material permits the liquid to travel through the emanator at a satisfactory travel rate. It will be appreciated, therefore, that the emanator may be comprised of any variety of suitable materials that provide the emanator with desired characteristics. In another exemplary embodiment of the invention, the emanator may comprise three or more materials, each of which contributes a desired characteristic to the emanator.

The materials of the emanator may be arranged in any suitable manner using any variety of suitable means that maintains the positioning of the materials relative to each other and does not adversely affect travel of liquid through the emanator. In one embodiment, the first material and second material may be disposed adjacent to each other, thereby forming an interface between the materials, but may be separated by a separating material or by space. Preferably, the first material and second material are disposed adjacent to each other and make contact with each other. More preferably, the first material and second material are arranged in such a maimer so as to maximize the amount of surface contact between the two materials.

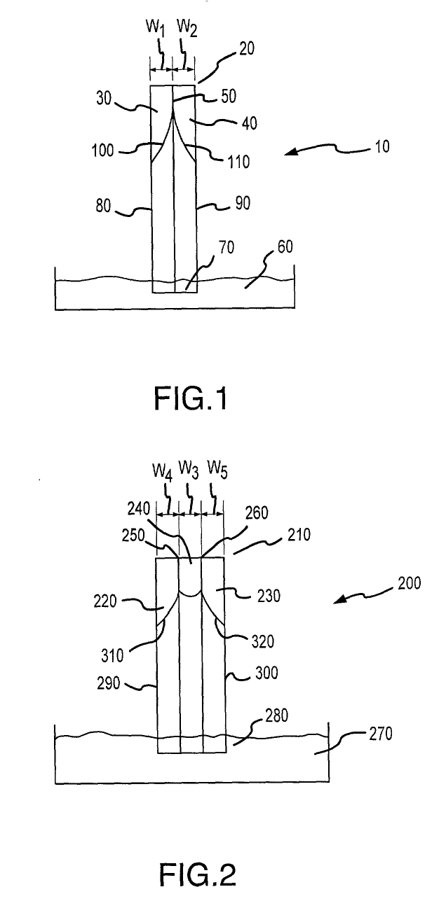

An exemplary embodiment of the present invention is illustrated in FIG. 1. An air freshener 10 includes an emanator 20 and a source of a liquid fragrance 60. As used herein, liquid fragrance may include components such as perfumes, oils, essential oils, botanical extracts, and/or any suitable solvents that may be used as carriers for the foregoing components. Emanator 20 is in fluid communication with the source of liquid fragrance 60. As illustrated by way of example in FIG. 1, emanator 20 has a first end 70 that contacts the liquid fragrance. It will be appreciated, however, that emanator 20 may contact the liquid fragrance in any suitable manner that effects wicking of the liquid fragrance within the emanator 20, as discussed in more detail below. The emanator 20 and source of liquid fragrance 60 may be original components of air freshener 10 or may be components of a refill used in air freshener 10.

Emanator 20 includes a first material 30 and a second material 40 which is disposed adjacent first material 30 so as to form an interface 50 between the materials. In one embodiment of the invention, first material 30 and second material 40 are separated, such as by a separation material or by space. In a preferred embodiment, first material 30 and second material 40 contact each other. As discussed in more detail below, interface 50 effects wicking of the liquid fragrance along emanator 20. Preferably, first material 30 and second material 40 are needle-punched together to ensure an effective interface 50 for wicking of the liquid fragrance. It will be appreciated, however, that any suitable mechanism may be used to maintain the positioning of the first material and the second material relative to each other as long as the mechanism does not adversely affect wicking of the liquid fragrance along interface 50.

In another exemplary embodiment of the invention, first material 30 and second material 40 each are made from a polymer material. Preferably, first material 30 and second material 40 are made from a nonwoven polymer material. The use of nonwoven polymer material enhances the migration of the liquid fragrance throughout each layer and reduces the likelihood that the liquid fragrance will be absorbed by the fibers of the material and become trapped. In addition, nonwoven polymer materials generally exhibit the ability to release the liquid fragrance to the environment without retaining excessive residual liquid fragrance. Examples of polymer materials that may be used to form first material layer 30 and second material layer 40 include polyethylene, polypropylene, polyester, nylon, and like materials. In one embodiment of the invention, first material 30 and second material 40 are formed from different polymer materials. In this manner, emanator 20 may be configured to take advantage of the properties of the two different materials. For example, the first polymer material may have more rigidity than the second polymer material but the second polymer material may more readily permit the liquid fragrance to evaporate. In a preferred embodiment of the invention, the first material 30 is formed of nylon. Nylon is not as flexible as polyethylene, polypropylene, and polyester and, accordingly, may contribute added rigidity to the emanator. In a more preferred embodiment of the invention, first material 30 is formed of nylon and second material 40 is formed of polypropylene. Liquid fragrance generally evaporates more readily from polypropylene than from nylon. An example of commercial material that may be used for emanator 20 and which has a nylon first material 30 needle-punched to a polypropylene second material 40 includes DS3605, which is sold by Reemay, Inc. of Old Hickory, Tenn. as a carpet-backing material.

When emanator 20 is used for evaporation of a liquid, it is brought in contact with a source of the liquid, such as the source of liquid fragrance 60. As illustrated in FIG. 1, a first end 70 of emanator 20 may be brought in contact with the source of liquid fragrance 60, although it will be appreciated that any suitable mechanism for contacting the liquid fragrance with emanator 20 may be used. FIG. 1 illustrates end 70 as being formed from the ends of both first material 30 and second material 40. Alternatively, end 70 may be formed of the end of either first material 30 or second material 40. With this configuration, for example, when end 70 is formed of the end of first material 30 only, the liquid fragrance may migrate through first material 30 to interface 50 and through second material 40.

Upon contact with emanator 20, the liquid fragrance may travel, or “wick”, along interface 50. In one embodiment of the invention, the liquid fragrance travels along the interface 50 at the same rate as it travels through either first material 30 or second material 40. In a preferred embodiment, interface 50 permits the liquid fragrance to travel along the emanator at a faster rate than it would be able to travel through either of the first material or the second material individually, as illustrated in FIG. 2. As the liquid fragrance travels along interface 50, it migrates through first material 30 to an opposing surface 80 of first material 30, as illustrated by flow line 100. The liquid fragrance may also migrate through second material 40 to an opposing surface 90 of second material 40, as illustrated by flow line 110. Depending on the composition of first material 30 and second material 40, the liquid fragrance in first material 30 may migrate to opposing surface 80 at the same travel rate as the liquid fragrance in the second material 40 migrates to opposing surface 90 (as when first material layer 30 and second material layer 40 are made of the same material) or may migrate at a different travel rate. As the liquid fragrance migrates to opposing surface 80 and/or 90, it evaporates from emanator 20. Again, depending on the composition of first material 30 and second material 40, the liquid fragrance may evaporate from each layer at an approximately equal rate or may evaporate more quickly from one of the materials. It will be appreciated that the volume of liquid fragrance that evaporates from each materials 30 and 40 is a function of the size of the surface area of each material. In other words, the greater the surface area of opposing surface 80 and a width W₁ of first material 30, the greater the volume of liquid fragrance that may evaporate from first material 30, and the greater the surface area of opposing surface 90 and a width W₂ of second material 40, the greater the volume of liquid fragrance that may evaporate from second material 40. In an alternative embodiment of the invention, the liquid fragrance may travel along interface 50 but may only evaporate from one of the materials 30 and 40.

FIG. 2 illustrates a further exemplary embodiment of the present invention. An air freshener 200 in accordance with this embodiment of the invention includes an emanator 210 and a source of liquid fragrance 270. Emanator 210 includes a first material 220 and a second material 230. A third material 240 is interposed between first material 220 and second material 230 so as to form a first interface 250 between the materials 220 and 240 and a second interface 260 between materials 230 and 240. In one embodiment, first material 220 and third material 240 are separated such as by a separation material or by space. In a preferred embodiment, first material 220 and third material 240 contact each other. Similarly, in one embodiment, second material 230 and third material 240 are separated by a separation material or space. In a preferred embodiment, second material 230 and third material 240 contact each other. First and second interfaces 250 and 260 effect wicking of the liquid fragrance along emanator 210. By utilizing two interfaces for the wicking of the liquid fragrance, emanator 210 is configured to permit a larger volume of liquid fragrance to evaporate from the emanator, thus increasing the efficacy of the air freshener. In one embodiment of the invention, the liquid fragrance travels along interface 250 and/or interface 260 at the same rate(s) as it travels through first material, second material and/or third material. In a preferred embodiment, interfaces 250 and 260 permit the liquid fragrance to travel along the emanator at a faster rate(s) than it would be able to travel through the first, second or third materials individually. It will be appreciated that any suitable mechanisms may be used to maintain the positioning of the first material, second material and third material relative to each other as long as the mechanisms do not adversely affect the wicking of the liquid fragrance along interfaces 250 and 260. Preferably, first material 220, second material 230 and third material 240 are needle-punched together.

In another exemplary embodiment of the invention, first material 220, second material 230, and third material 240 each are made from a polymer material. Preferably, first material 220, second material 230 and third material 240 are made from nonwoven polymer material. As described above, the use of nonwoven polymer material enhances the migration of the liquid fragrance throughout each layer and reduces the likelihood that the liquid fragrance will be absorbed by the fibers of the material and become trapped. In addition, nonwoven polymer materials generally exhibit the ability to release the liquid fragrance to the environment without retaining excessive residual liquid fragrance. Examples of nonwoven polymer materials that may be used to form first material 220, second material 230 and third material 240 include polyethylene, polypropylene, polyester, nylon, and like materials. In one embodiment of the invention, third material 240 may be formed of polymer material that is different from first material 220 and second material 230. In this manner, third material 240 may be used to contribute structural integrity to emanator 210. For example, the material that forms third material 240 may have more rigidity than the polymer material(s) that form first material 220 and second material 230. In a preferred embodiment of the invention, third material 240 is formed of nylon. Nylon is not as flexible as polyethylene, polypropylene, and polyester and, accordingly, may contribute added rigidity to emanator 210. In more preferred embodiment of the invention, first material 220 and second material 230 are formed of polypropylene and third material 240 is formed of nylon.

When emanator 210 is used for evaporation of a liquid, it is brought in contact with a source of the liquid, such as the source of liquid fragrance 270. As illustrated in FIG. 2, a first end 280 of emanator 210 may be brought in contact with the source of liquid fragrance 270, although it will be appreciated that any suitable mechanism for contacting the liquid fragrance with emanator 210 may be used. FIG. 2 illustrates first end 280 as being formed from the ends of first material 220, second material 230 and third material 240. Alternatively, first end 280 may be formed of the end of only one of materials 220, 230 and 240. With this configuration, for example, when first end 280 is formed of the end of first material 220, the liquid fragrance may migrate through first material 220 to interface 250, through third material 240 to interface 260, and through second material 230.

Upon contact with emanator 210, the liquid fragrance wicks along interfaces 250 and 260. Preferably, interfaces 250 and 260 permit the liquid fragrance to travel along the emanator at a faster rate(s) than it would be able to travel through first material 220, second material 230 or third material 240, individually. As the liquid fragrance travels along interface 250, it migrates to an opposing surface 290 of first material 220, as illustrated by flow line 310, and migrates through third material 250. As the liquid fragrance travels along interface 260, it migrates to an opposing surface 300 of second material 230, as illustrated by flow line 320, and also migrates through third material 240. Depending on the composition of first material 220 and second material 230, the liquid fragrance in first material layer 220 may migrate to opposing surface 290 at the same travel rate as the liquid fragrance in the second material 230 migrates to opposing surface 300 (as when first material 220 and second material 230 are made of the same material) or may migrate at a different travel rate. As the liquid fragrance migrates to opposing surface 290 and/or 300, it evaporates from emanator 210. Again, depending on the composition of first material 220 and second material 230, the liquid fragrance may evaporate from each material at an approximately equal rate or may evaporate more quickly from one of the materials. The liquid fragrance may also evaporate from the perimeter surfaces of third material 240 that are exposed to the atmosphere. The volume of liquid fragrance that evaporates from third material 240 is a function of the width W₃ of these perimeter surfaces of third material 240.

It will be appreciated that the volume of liquid fragrance that evaporates from each material 220 and 230 is a function of the size of the surface area of each material exposed to the environment, in addition to other features such as orientation and configuration of each material. In other words, the greater the surface area of opposing surface 290 and a width W₄ of first material 220, the greater the volume of liquid fragrance that evaporates from first material 220, and the greater the surface area of opposing surface 300 and a width W₅ of second material 230, the greater the volume of liquid fragrance that evaporates from second material 230.

In the foregoing specification, the invention has been described with reference to specific embodiments. However, it will be understood by those skilled in the art that various changes may be made and equivalents may be substituted for elements thereof without departing from the scope of the invention. In addition, many modifications may be made to adapt to a particular situation or material to the teachings of the invention without departing from the essential scope thereof. Therefore, it is intended that the invention not be limited to any particular embodiment disclosed for carrying out this invention, but that the invention includes all embodiments falling within the scope of the appended claims.

Benefits, other advantages, and solutions to problems have been described above with regard to specific embodiments. However, the benefits, advantages, solutions to problems, and any element(s) that may cause any benefit, advantage, or solution to occur or become more pronounced are not to be construed as a critical, required, or essential features or elements of any or all the claims. As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

We claim:

1. An emanator for evaporation of a liquid therefrom, the emanator comprising:
a first material; and
a second material that is disposed adjacent to said first material;
wherein the emanator is configured such that a liquid travels through the emanator at a rate no less than a rate at which it would travel through said first material alone and no less than a rate at which it would travel through said second material alone.

2. The emanator of claim 1, wherein the emanator is configured such that said liquid travels through the emanator at a rate faster than a rate at which it would travel through said first material alone and faster than a rate at which it would travel through said second material alone.

3. The emanator of claim 1, wherein said first material contacts said second material.

4. The emanator of claim 1, said first material comprising at least one of polyethylene, polypropylene, polyester and nylon.

5. The emanator of claim 1, said second material comprising at least one of polyethylene, polypropylene, polyester and nylon.

6. The emanator of claim 1, said first material comprising nylon and said second material comprising polypropylene.

7. The emanator of claim 1, wherein said first material and said second material are needle-punched together.

8. The emanator of claim 1, the liquid comprising liquid fragrance.

9. An air freshener comprising:
a source of a liquid fragrance; and
an emanator in fluid communication with said source of said liquid fragrance, said emanator comprising:
a first material through which said liquid fragrance has a first travel rate; and
a second material through which said liquid fragrance has a second travel rate, said second material contacting said first material;
wherein the emanator is configured such that said liquid fragrance has a third travel rate through the emanator, said third travel rate no less than said first travel rate and said second travel rate.

10. The air freshener of claim 9, wherein the emanator is configured such that said third travel rate is greater than said first travel rate and said second travel rate.

11. The air freshener of claim 9, said first material comprising at least one of polyethylene, polypropylene, polyester and nylon.

12. The air freshener of claim 9, said second material comprising of at least one of polyethylene, polypropylene, polyester and nylon.

13. The air freshener of claim 9, said first material comprising nylon and said second material comprising polypropylene.

14. The air freshener of claim 9, wherein said first material and said second material are needle-punched together.

15. An emanator for evaporation of a liquid therefrom, the emanator comprising:
a first material;
a second material adjacent to said first material and forming a first interface with said first material;
wherein the emanator is configured to permit a liquid to travel along said first interface and to evaporate from at least one of said first material and said second material.

16. The emanator of claim 15, the emanator further comprising:
a third material adjacent to said second material and forming a second interface with said second material;
wherein the emanator is configured to permit said liquid to travel along said second interface and to evaporate from at least one of said second material and said third material.

17. The emanator of claim 15, said first material comprising at least one of polyethylene, polypropylene, polyester and nylon.

18. The emanator of claim 15, said third material comprising at least one of polyethylene, polypropylene, polyester and nylon.

19. The emanator of claim 15, said first material and said third material comprising polypropylene and said second material comprising nylon.

20. The emanator of claim 15, wherein said first material, said second material and said third material are needle-punched together.

21. The emanator of claim 15, where said first material contacts said second material.

22. The emanator of claim 16, wherein said third material contacts said second material.

23. A refill for an air freshener, the refill comprising:
a source of a liquid fragrance; and
an emanator in contact with said source of said liquid fragrance, said emanator comprising:
a first material; and
a second material contacting said first material;
wherein the emanator is configured such that said liquid fragrance travels through said emanator at a rate no less than a rate at which it would travel through said first material alone and no less than a rate at which it would travel through said second material alone.

24. The refill of claim 23, wherein the emanator is configured such that said liquid fragrance travels through said emanator at a rate faster than a rate at which it would travel through said first material alone and faster than a rate at which it would travel through said second material alone.
